(19) United States
(12) Patent Application Publication
(10) Pub. No.: US 2022/0106869 A1
(43) Pub. Date: Apr. 7, 2022

(54) BREAKDOWN PRESSURE REDUCTION BASED ON INJECTION OF COOLING AGENTS

(71) Applicant: Saudi Arabian Oil Company, Dhahran (SA)

(72) Inventors: Kaiming Xia, Dhahran (SA); Tariq Mahmood, Dhahran (SA); Saidi Ali Hassani, Dhahran (SA)

(21) Appl. No.: 17/062,346

(22) Filed: Oct. 2, 2020

(51) Int. Cl. E21B 47/07; E21B 36/00; E21B 49/00; E21B 43/26; G01V 99/00; G01N 33/24

(52) U.S. Cl. CPC E21B 47/07; E21B 36/001; E21B 49/006; E21B 49/02; G01V 99/005; G01N 33/24; E21B 43/26

(57) ABSTRACT

Systems and methods are related to techniques related to reducing breakdown pressure for open hole hydraulic fracturing treatment through injecting cooling agents. The techniques may include identifying a stage of a multi-stage borehole. The techniques may further include identifying a parameter of rock that is adjacent the multi-stage borehole. The techniques may further include identifying, based on the parameter, a criteria of a cooling process that is to be performed on the rock. As a result, breakdown pressure of the rock may be reduced. The techniques may further include outputting an indication of the criteria for use in performing the cooling process. Other embodiments may be described or claimed.

### BACKGROUND

The present disclosure applies to open-hole hydraulic fracturing treatment. For deep and tight gas reservoirs, horizontal wells may be drilled for large reservoir contact. In these applications, transverse hydraulic fractures may be desired, which suggest that the horizontal part of the wells should be drilled in the minimum horizontal stress direction. However, in this application, formation breakdown for initiating transverse fractures may be a challenging issue for zonal isolated open-hole multi-stage hydraulic fracturing treatment.

### SUMMARY

As noted, rock breakdown or fracture initiation should be firstly achieved for a successful hydraulic fracturing treatment. For the hydraulic fracturing treatment, reliable breakdown of the near-borehole formation may affect the selection of right casing size, tubing size, wellhead, and burst pressure limits, as well as pump schedule design. Hydraulic fracturing pump schedule may not be able to be injected as planned if breakdown is an issue (i.e., if breakdown of a particular stage along the wellbore may not be achieved based on a planned hydraulic fracturing injection).

Additionally, wellheads of a wellbore (e.g., an entrance portion of the wellbore) may have a pressure limit. In some embodiments, the pressure limit may be approximately 15,000 pounds per square inch (psi). For some deep and tight gas reservoirs, breakdown may be a challenge because it is difficult to get a high enough pressure acting on the wellbore without exceeding the wellhead pressure limit.

The present disclosure describes techniques that can be used for hydraulic fracturing treatment, especially for open-hole multi-stage hydraulic fracturing treatment. Specifically, embodiments relate to an approach to reduce the breakdown pressure through cooling the borehole first, which may be achieved by injecting cooling agents and flow to the target open-hole fracturing interval. As a result, the cooling agent may lead to a temperature drop in the near-borehole formation. As will be described in greater detail below, the cooling agents may be or include one or more of dry ice, ethanol liquid nitrogen, water ice, slick water, etc. Simultaneously, the near-wellbore formation may exhibit thermal contraction and tensile stress increase in the borehole direction. Also, the thermal contraction leads to compression hoop stress increase, which can inhibit the longitudinal fracture initiation. Eventually, the breakdown pressure along the wellbore axis direction may be reduced, and facilitate the initiation of transverse hydraulic fractures for open-hole hydraulic fracturing treatment.

In some embodiments, the required axial tensile stress changes along the borehole axis may vary from well to well and reservoir to reservoir. For accurately estimating the required axial stress change along the borehole axis, a three-dimensional (3D) fully-coupled thermal-displacement finite element model may be used to facilitate the cooling agent injection design. Embodiments herein may relate to the development and use of such a model, as well as the criteria to terminate the cooling process.

In some implementations, a computer-implemented method may include identifying a stage of a multi-stage borehole. The method may further include identifying a parameter of rock that is adjacent the multi-stage borehole. The method may further include identifying, based on the parameter, a criteria of a cooling process that is to be performed on the rock. The method may further include outputting an indication of the criteria for use in performing the cooling process. The cooling process may, for example, reduce breakdown pressure of the rock in the multi-stage borehole.

The previously described implementation is implementable using a computer-implemented method; a non-transitory, computer-readable medium storing computer-readable instructions to perform the computer-implemented method; and a computer-implemented system including a computer memory interoperably coupled with a hardware processor configured to perform the computer-implemented method/the instructions stored on the non-transitory, computer-readable medium.

The subject matter described in this specification can be implemented in particular implementations, so as to realize one or more of the following advantages. First, embodiments may help reduce the breakdown pressure within a borehole, and improve the success rate of breakdown for hydraulic fracturing treatments. Specifically, embodiments may present an efficient method to reduce the required breakdown pressure, which may be desirable for successful initiation of hydraulic fracture for open-hole multi-stage fracturing. Embodiments may also provide a technique, system, or criteria related to modeling a cooling process such that the cooling process may be performed in a controlled and efficient manner.

The details of one or more implementations of the subject matter of this specification are set forth in the Detailed Description, the accompanying drawings, and the claims. Other features, aspects, and advantages of the subject matter will become apparent from the Detailed Description, the claims, and the accompanying drawings.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 depicts an example of a cooling process within a multi-stage borehole, in accordance with various embodiments.

FIG. 2A depicts an example of a thermal-displacement finite element model which may be used in simulations of the cooling process, in accordance with various embodiments.

FIG. 2B depicts an example of a transient temperature contour around the borehole during a cooling process, in accordance with various embodiments.

FIG. 3 depicts an example of a temperature gradient contour around the borehole during a cooling process, in accordance with various embodiments.

FIG. 4 depicts an example of induced axial stress along the axis of a borehole during a cooling process, in accordance with various embodiments.

FIG. 5 depicts a high-level example technique of performance of a cooling process, in accordance with various embodiments.

FIG. 6 depicts an alternative high-level example technique of performance of an aspect of a cooling process, in accordance with various embodiments.

FIG. 7 is a block diagram illustrating an example computer system used to provide computational functionalities associated with described algorithms, methods, functions, processes, flows, and procedures as described in the present disclosure, according to some implementations of the present disclosure.

Like reference numbers and designations in the various drawings indicate like elements.

### DETAILED DESCRIPTION

The following detailed description describes systems and techniques for cooling a stage of a multi-stage borehole. Various modifications, alterations, and permutations of the disclosed implementations can be made and will be readily apparent to those of ordinary skill in the art, and the general principles defined may be applied to other implementations and applications, without departing from scope of the disclosure. In some instances, details unnecessary to obtain an understanding of the described subject matter may be omitted so as to not obscure one or more described implementations with unnecessary detail and inasmuch as such details are within the skill of one of ordinary skill in the art. The present disclosure is not intended to be limited to the described or illustrated implementations, but to be accorded the widest scope consistent with the described principles and features.

As noted, embodiments relate to a cooling process within an open borehole multi-stage hydraulic fracturing treatment. In the borehole, the horizontal portion of the borehole may be generally aligned at the minimum horizontal stress direction for generating transverse hydraulic fractures. Assuming the horizontal part landing in either normal fault or strike-slip regimes, the stress state around the borehole may be estimated based on the logging data and linear elasticity.

From a mechanics point of view, the bottom hole pressure from hydraulic fracturing fluid injection may not efficiently induce tensile stress along the borehole axis direction so that transverse hydraulic fractures can be initiated. Additionally, the induced axial stress may be very low (i.e., approaching zero) compared to the hoop stress if the borehole surface is smooth and the borehole axis aligns at the minimum horizontal stress direction. Also it may satisfy the ideal plane strain condition. In this situation, the induced tensile stress maybe appreciable only if there are notches existing on the zonal isolated borehole wall, and little to no hydraulic fluid leaks off into the borehole formation. Otherwise, longitudinal hydraulic fractures may initiate firstly, which may be against the goal of hydraulic fracturing treatment for transverse hydraulic fractures. To address this issue, embodiments herein relate to techniques to reduce breakdown pressure prior to injecting the pump schedule of the main hydraulic fracturing treatment.

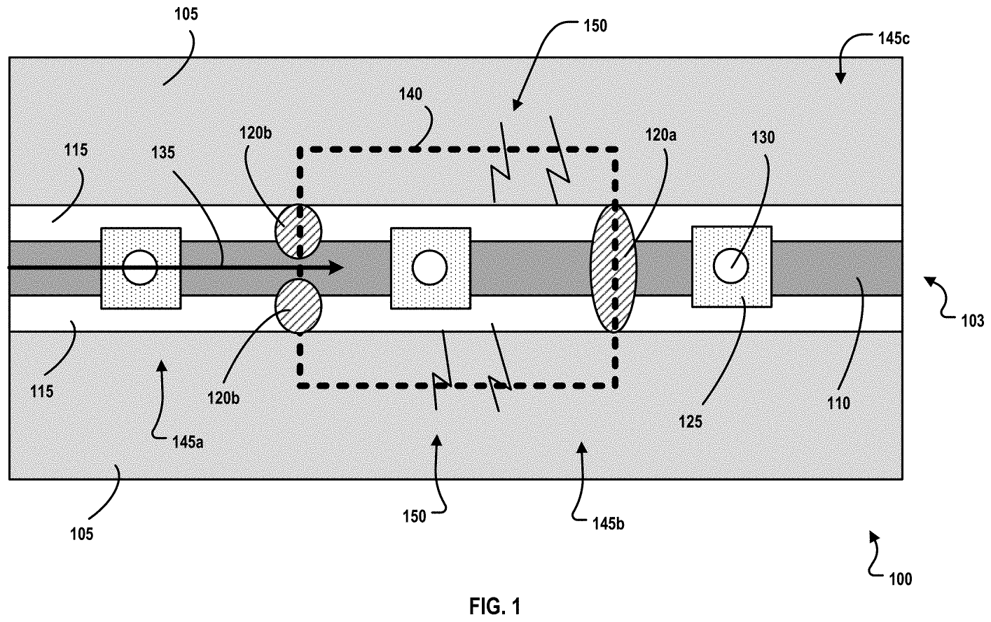

FIG. 1 depicts an example of a cooling process within a multi-stage wellbore 100, in accordance with various embodiments. It will be understood that the depiction of FIG. 1 is intended as a simplified cross-sectional view of the multi-stage wellbore 100. Specifically, FIG. 1 depicts a generally horizontal wellbore 100, which has a tubing 110 in an open borehole 103 within rock 105. Although the wellbore 100 and borehole 103 are depicted and described as generally horizontal, in some embodiments the wellbore 100 and borehole 103 may have an angle of approximately +/−10 degrees from horizontal, although in other embodiments the angle may be slightly more or less dependent on the specific embodiment.

The tubing 110 may be at least partially surround by an annulus 115 within the borehole 103 as shown in FIG. 1. Specifically, the annulus 115 may be a spacing between the tubing 110 and the rock 105 as shown in FIG. 1. It will be understood that although the annulus 115 is depicted as being generally empty and the lines of both the tubing 110 and the rock 105 are depicted as being straight/smooth, in real-world embodiments the lines of one or both of the tubing 110 or the rock 105 may have imperfections. Additionally, there may be one or more elements positioned within the annulus 115 such as return-lines, etc. Other variations may be present.

The multi-stage wellbore 100 may include a number of stages such as stages 145a, 145b, and 145c. It will be understood that, although only three stages 145a/145b/145c are explicitly described herein, in other embodiments the wellbore 100 may include more or fewer stages. For example, in some embodiments, the multi-stage wellbore 100 may have up to approximately 30 or more stages, although in other embodiments the number of stages may be higher or lower.

For open-hole hydraulic fracturing treatment, the fracturing stages 145a/145b/145c may be separated by an element such as an open-hole isolated packer 120a and 120b. The packer 120a/120b may be an element that is expandable, for example through mechanical or hydraulic means, to create a seal or blockage within the tubing 110. As shown in FIG. 1, the packer 120a is shown in a “closed” or “sealed” configuration such that hydraulic fluid, cooling agent, etc. may not travel through the tubing 110 from stage 145b to stage 145c. By contrast, packer 120b is shown in an “open” or “unsealed” configuration such that hydraulic fluid, cooling agent, or some other material may travel through the tubing 110 from stage 145a to stage 145b. Although packer 120b is shown as having an opening generally in a central location of the packer 120b, in other embodiments the packer 120b may be located at a periphery of the borehole 103 and configured to extend to an opposite edge of the borehole 103, may be configured in a “flap” type configuration, or have some other configuration.

The multi-stage wellbore 100 may further include a number of junctions 125 with ports 130. Specifically, the port 130 may be selectively opened or closed so that fluid (e.g., hydraulic fracturing fluid) may be ejected from the port 130 to the surrounding rock 105 to perform hydraulic fracture of the rock 105. In some embodiments, the port 130 may be configured such that a ball is seated into the port 130. High pressure hydraulic fluid may exit the fluid port 130 and cause fracturing. Backflow of the high pressure fluid may be prevented by packers 120, which may be expanded to effectively seal a stage in which the hydraulic fracturing treatment is occurring. In some embodiments, each stage 145a/145b/145c may include a single junction 125 and port 130, while in other embodiments respective ones of the stages 145a/145b/145c may include a plurality of junctions 125 and/or ports 130, or may not include a junction 125 or port 130.

In hydraulic fracturing operation, it may be desirable to initiate a transverse hydraulic fracture of the rock 105 at stage 145b. Such a fracture is indicated by the jagged lines 150 extending from the open borehole 103 into the rock 105 at stage 145b. However, as noted, it may be difficult to initiate such a fracture because the breakdown pressure required to fracture the rock 105 at stage 145b may require a pressure at the wellhead that is greater than the pressure limit that the wellhead is designed to sustain (e.g., greater than approximately 15,000 psi at the wellhead). Further, it may not be efficient to generate a high axial tensile stress along the borehole direction based on the fundamental principle of mechanics for an open borehole condition. Rather than it may be more efficient to induce a high tensile hoop stress in this situation.

To reduce this breakdown pressure so that transverse fracture can be initiated, a cooling agent may be provide along axis 135 of the borehole 103. More specifically, the cooling agent may be injected through downhole tubing 110 and flow out of the open port into the annulus 115 for the open-hole interval for stage 145b. Packer 120b may then be sealed so that the cooling agent is sealed inside stage 145b and cools the rock 105 within zone 140. It will be understood that the demarcation of zone 140 is intended purely for the sake of example and discussion, and in real-world embodiments the cooling affected by the cooling agent may extend in one or more directions beyond the boundary indicated by the demarcation of zone 140, in a uniform or non-uniform ways, which may be dependent on the thermal mechanical property of rock, porosity, permeability, and injecting cooling agent, etc.

Once injected into stage 145b, the cooling agent may directly interface with the borehole 103 and, more specifically, the rock 105 surrounding the borehole 103, and cool the zone 140. Then, thermal contraction may occur along the borehole axis direction (e.g., in a direction parallel to axis 135), and simultaneously induce tensile stress within zone 140 (e.g., stress caused by contraction of the rock 105 in a direction parallel to axis 135. The increase in tensile stress of the rock 105 may reduce the breakdown pressure (i.e., the pressure of hydraulic fluid required to initiate transverse fractures of the rock). Also cooling the borehole may lead to a contraction in the radial direction, which in turn may lead to an increasing compression hoop stress. Increasing hoop stress in compression may lower the chance to initiate longitudinal fractures, which is strongly desired for hydraulic fracturing horizontal wells.

Generally, the cooling rate (e.g., the cooling agent(s) used, the length of time the cooling agent(s) are applied, etc.) may be based on numerical modeling and thermal energy transfer/conversion as will be described in greater detail below. It will be noted that although the cooling process is only described with respect to stage 145b, in some embodiments the cooling process may be executed for a number of stages, either sequentially or simultaneously. In some embodiments, one may execute the cooling process for stages sequentially or alternatively so that the cooling process may be tuned to achieve a specific amount of cooling based on parameters of the rock that is being cooled, which may vary from stage to stage. The cooling sequences may be optimized based on factors such as stress shadow.

To simulate the thermal mechanics problem, some material properties or parameters may be evaluated. Such parameters may include, for example, reservoir temperature of a gas reservoir within the rock 105. The reservoir temperature may be measured through a downhole logging device. Other parameters may include, for example, rock thermal properties such as thermal expansion or thermal conductivity of the rock 105, specific heat, etc. These parameters may be based on tests of a sample of the rock 105, such as may be performed in a lab. Mechanical properties of the rock 105 may be extracted through processing of a sonic log of the rock 105, lab tests of the rock 105, etc. Such mechanical properties may include, for example, the Young's modulus of the rock 105, the Poisson ratio of the rock 105, the density of the rock 105, etc.

The cooling process, or simulations thereof, may be based on one or more of the above-listed properties of the rock 105 or reservoir. Specifically, the type of cooling agent, or the time with which the cooling agent is applied, may be based on one or more of the above properties.

In some embodiments, the cooling agent may be or include dry ice. Dry ice is typically a solid form of carbon dioxide (CO₂) which has a lower temperature than that of water ice. Dry ice sublimates at −78.5 degrees Celsius (° C.) at Earth atmospheric pressures. Additionally or alternatively, the cooling agent may be or include liquid nitrogen (N₂), which has a boiling point at sea level of approximately −195.79° C., and a freezing point at approximately −210° C. Additionally or alternatively, the cooling agent may be or include ethanol (C₂H₅OH) which has a freezing point of approximately −114.14° C. and a boiling point of approximately 78.5° C., and which may be used in conjunction with dry ice as a cooling agent. Typically, ethanol may be a colorless, low-viscosity liquid which may be appropriate for use as a coolant. Therefore, dry ice, ethanol, liquid nitrogen, and water ice (H₂O) may be potential materials for use as cooling agents in a stage of a borehole such as stage 145b of borehole 103. As previously noted, the specific mixture or selection of the various cooling agents may be calculated based on the required temperature drop of the rock 105 to affect the breakdown pressure, the cooling process itself, thermal energy equations of the model, and rock thermal or mechanical properties.

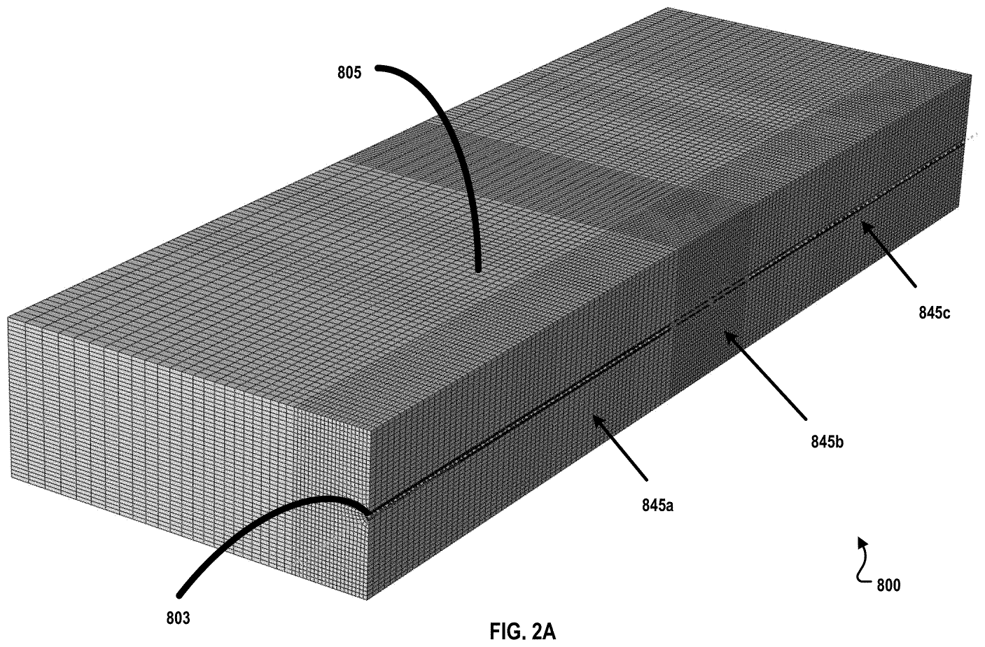

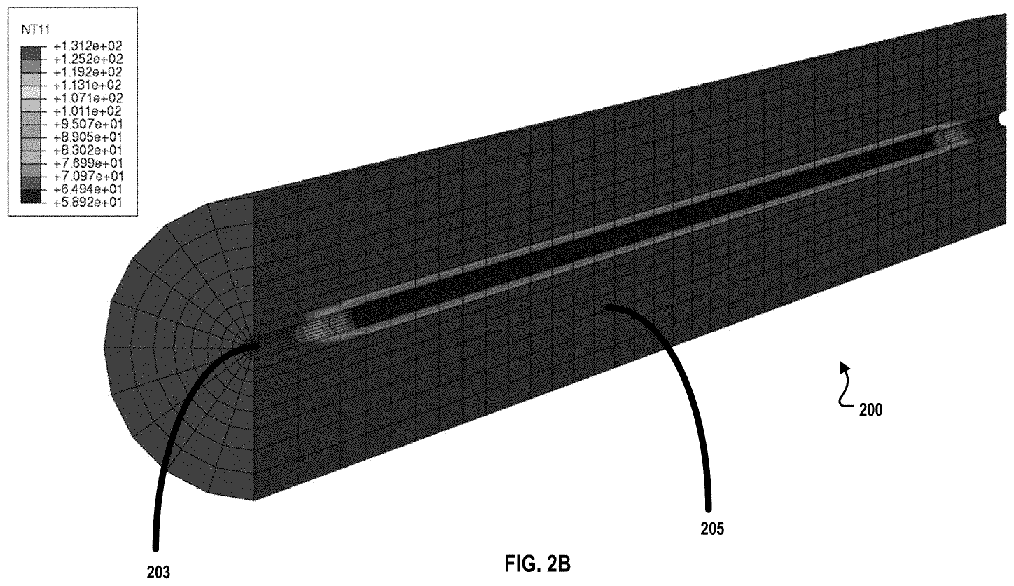

To estimate the cooling process, a fully-coupled thermal-displacement finite element model may be used. Specifically, the model may be used to predict the transient temperature drop of the rock 105, stress change(s) of the rock 105, etc. Certain visual representations of the model are depicted in FIGS. 2A, 2B, 3, and 4. It will be understood that, although certain of the models depict specific numerical ranges, these ranges are only depicted for the sake of explanation and discussion. Other embodiments may have different ranges, units, degrees of accuracy, etc. based on various factors such as the characteristics of the rock, the cooling agent, the accuracy or settings of the model, the sample size associated with the rock, etc.

FIG. 2A depicts an example of a finite element model 800 which depicts the interface between the borehole and the cooling agent. Specifically, the model 800 may depict rock (which may be similar to rock 105) with a borehole 803 therein (which may be similar to borehole 103). The borehole may have 3 stages 845a, 845b, and 845c, which may be respectively similar to stages 145a, 145b, and 145c. In the depicted model 800, the stage 145b may be undergoing the cooling process. Specifically, a cooling agent may be positioned within the borehole 803 at stage 845b. The heat may be transferred from the relatively higher-temperature reservoir or rock 805 at the stage 845b to the lower temperature cooling agent within the borehole 803 through surface film condition, which is defined with a film coefficient that has units Watts per meter squared per degree Celsius (W/m²/° C.), sink temperature, and its distribution type. Another graphical representation may include a representation of the temperature contour for all or part of the reservoir model, not shown for the sake of redundancy of other models depicted herein.

FIG. 2B depicts an example of a transient temperature contour around the borehole during a cooling process, in accordance with various embodiments. Specifically, the model 200 of FIG. 2B depicts the transient temperature of rock 205 and a borehole 203, which may be respectively similar to rock 105 and borehole 103. The legend associated with FIG. 2B depicts the transient temperature of the model in units of ° C. As may be seen, the temperature of the rock 205 may be approximately 131.2° C., while the temperature of the borehole 203 may be approximately 58.92° C. based on the presence of the cooling agent. The temperature along the periphery of the borehole 203 may vary between the high ends and the low ends of those ranges as may be seen.

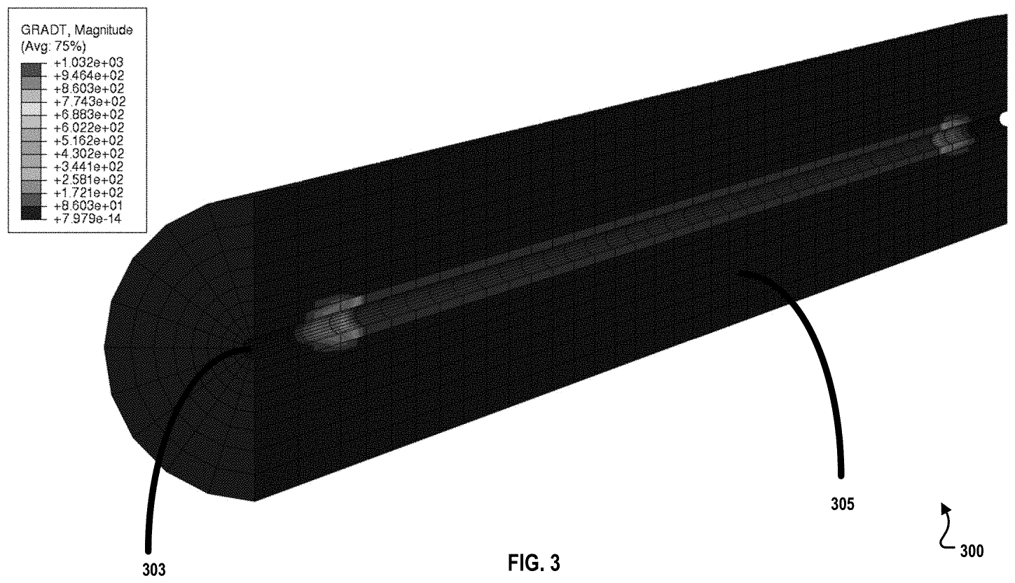

FIG. 3 depicts an example of a temperature gradient contour around the borehole during a cooling process, in accordance with various embodiments. Specifically, the model 300 of FIG. 3 depicts the temperature gradient of rock 305 and borehole 303, which may be respectively similar to rock 105 and borehole 103. The legend of FIG. 3 is depicted in units of ° C./m. As may be seen, the temperature gradient may be relatively sharp (e.g., exhibit a significant amount of change) around the borehole 303 due to the cooling agent being present in the borehole 303.

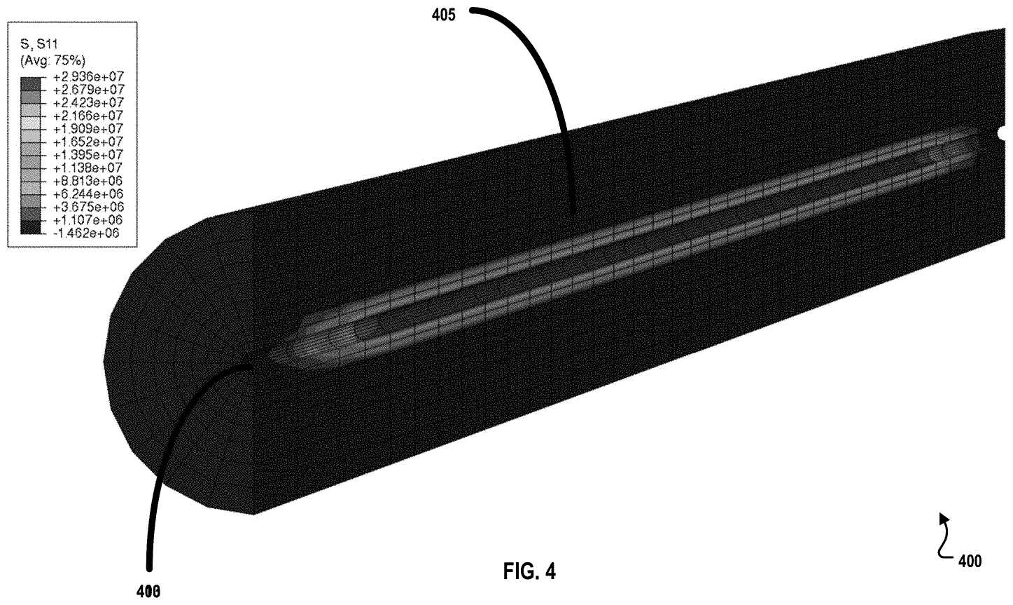

FIG. 4 depicts an example of induced axial stress along the axis of a borehole during a cooling process, in accordance with various embodiments. Specifically, the model 400 of FIG. 4 depicts the axial stress along rock 405 and borehole 403, which may be respectively similar to rock 105 and borehole 103. The legend of FIG. 4 is depicted in units of Pascals. As may be seen in FIG. 4, the axial stress at the borehole 403 may be approximately 30 Megapascals (MPa). By contrast, the axial stress at the rock 405 may be approximately 1.5 MPa (negative sign means compression stress). This change in axial stress between the rock 405 and the borehole 403 may represent enough of a change to significantly reduce the breakdown pressure for initiating transverse hydraulic fractures within the rock 405.

Based on the finite element modeling, e.g., the models depicted in FIGS. 2A, 2B, 3, and 4, one may be able to identify aspects of the cooling process such as the transient cooling process, temperature drop, or stress change. These aspects may be used to identify criteria to terminate the cooling process. For example, the criteria to terminate the cooling process may be a time span in which the cooling process should be performed. In some embodiments, the cooling process may take on the order of 10 minutes, while in other embodiments the cooling process may be longer or shorter. For example, in some embodiments the cooling process may be performed for between approximately 10 minute and approximately 20 minutes. Typically, the amount of time for the cooling process may be based on one or more factors such as thermal mechanical properties of the rock, temperature of the rock, temperature of the coolant, etc.

In one embodiment, the criteria to terminate the cooling process may be based on the induced axial tensile stress change along the borehole axis, which may allow the rock to break down without exceeding the pressure limit at the wellhead. For open-hole hydraulic fracturing and well horizontal part aligning at the minimum horizontal stress direction, the cooling process may be stopped when the induced axial tensile stress meets the following condition: σ_{T_axial}=T+σ_{Hmin}. In this embodiment, σ_{T_axial} may represent the required or predefined change in induced axial tensile stress. T may refer to the tensile strength of the formation of the borehole where the transverse hydraulic fracture is desired. σ_{Hmin} may denote the effective in-situ minimum horizontal stress. Generally, even for a perforation hydraulic fracturing method, it may be desirable to increase the induced axial tensile stress (e.g., σ_{T_axial}) to a threshold value which may allow bottom hole pressure to break down the rock around the perforation tunnel, while keeping the surface pressure below the wellhead pressure limit. Once this induced axial tensile stress is achieved, the cooling process may be terminated, and injection of the main pump schedule for hydraulic fracturing may be started.

In case the horizontal part of borehole is not at the minimum horizontal stress direction, the σ_{Hmin} will be replaced by the in-situ stress projected onto the borehole direction, which is named by σₐₓᵢₐₗ^{In-situ}.

In order to obtain the in-situ stress σ_{Pr} projected onto the axial direction of borehole σₐₓᵢₐₗ^{In-situ}, two coordinate systems may be established: global coordinate system (x_{G}y_{G}z_{G}) and borehole coordinate system (x_{B}y_{B}z_{B}). The global coordinate system is defined as the x-axis always aligning with the North, y-axis aligning with the East and therefore the z-axis is vertically downwards. Generally, one may assume the azimuth of maximum principal stress is θ_{σH},which is the angle turning clockwise from North to the maximum principle stress. Therefore the rotation matrix is given by

$${R}_{{σ}_{H} \to G} = ( \begin{matrix}cos {θ}_{{σ}_{H}} & - sin {θ}_{{σ}_{H}} & 0 \\ sin {θ}_{{σ}_{H}} & cos {θ}_{{σ}_{H}} & 0 \\ 0 & 0 & 1\end{matrix} )$$

Generally, θ_{σH} should be positive if the maximum principal stress σ_{Hmax} is clockwise to the axis-x_{G}, and negative if it is counter clockwise to the axis-x_{G}. For well survey, any point along the well trajectory may be determined by three parameters: MD (measured depth), α_{D}(wellbore deviation), and α_{A}(wellbore azimuth). The borehole coordinate system at any point along the well trajectory may be tracked and obtained by the following rotations about the global coordinate system x_{G}y_{G}z_{G} particularly following two steps: (1) rotation of deviation α_{D} about the y_{G}-axis; and (2) rotation of azimuth α_{A} about z_{G}-axis. Then the transformation matrix is given by

$${R}_{G \to B} ( {α}_{A} , {α}_{D} ) = {R}_{y} ( {α}_{D} ) {R}_{z} ( {α}_{A} ) = ( \begin{matrix}cos {α}_{D} & 0 & - sin {α}_{D} \\ 0 & 1 & 0 \\ sin {α}_{D} & 0 & cos {α}_{D}\end{matrix} ) ( \begin{matrix}cos {α}_{A} & sin {α}_{A} & 0 \\ - sin {α}_{A} & cos {α}_{A} & 0 \\ 0 & 0 & 1\end{matrix} )$$

Then in-situ stress tensor can be projected onto the borehole coordinate system by

σ^{B,I}=R_{G→B}(α_{C},α_{A})R_{σH→G}σ_{Pr}R_{G→B}^{T}(α_{D},α_{A})

σₐₓᵢₐₗ^{In-situ} is the stress component at the direction of z-axis of the borehole coordinate system, which is calculated using the above equation.

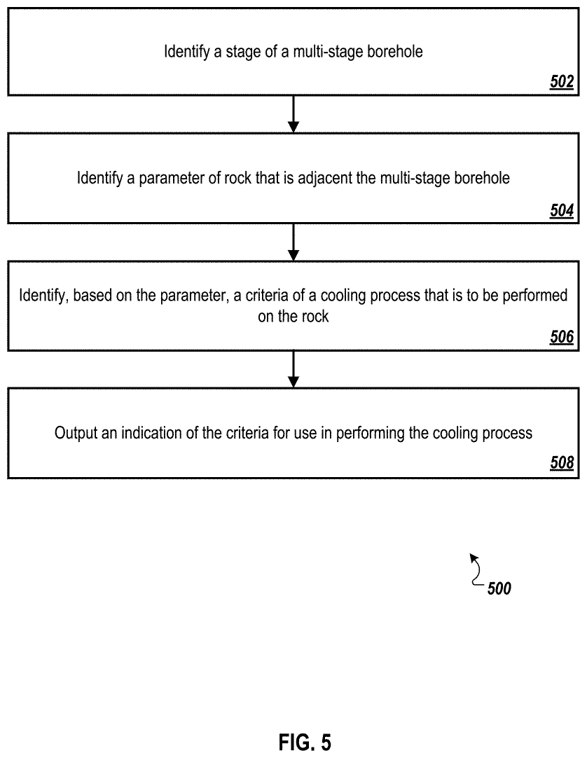

FIG. 5 depicts a high-level example technique 500 of performance of a cooling process, in accordance with various embodiments.

The technique may include identifying, at 502, a stage of a multi-stage borehole. The stage may be similar to, for example, stage 145b of borehole 103.

The technique may further include identifying, at 504, a parameter of rock (e.g., rock 105) that is adjacent to the multi-stage borehole. The parameter may be or may include, for example, parameter of the rock is related to thermal conductivity of the rock, thermal expansion of the rock, specific heat of the rock, Young's modulus related to the rock, Poisson ratio related to the rock, or density of the rock.

The technique may further include identifying, at 506 based on the parameter, a criteria of a cooling process that is to be performed on the rock. The criteria may be, for example, a type of cooling agent to be used, a time period over which the cooling process should be performed, a desired change in axial tensile stress (e.g., the value of σ_{T_axial}), etc.

The technique may further include outputting, at 508, an indication of the criteria for use in performing the cooling process. The parameter may be output as, for example, an indication in a user interface (UI), an indication on a model such as the models shown in FIGS. 2A, 2B, 3, and 4, or some other indication which may be provided to a user.

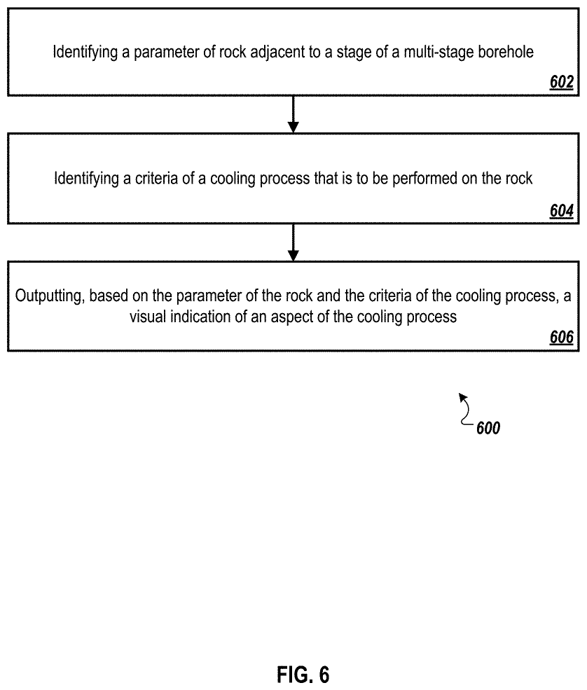

FIG. 6 depicts an alternative high-level example technique 600 of performance of an aspect of a cooling process, in accordance with various embodiments. The technique may include identifying, at 602, a parameter of rock adjacent to a stage of a multi-stage borehole. The rock may be, for example rock 105. The stage may be, for example, stage 145b. The borehole may be, for example borehole 103. The parameter may be, for example, one or more of the parameters described with respect to element 504 or elsewhere herein.

The technique may further include identifying, at 604, a criteria of a cooling process that is to be performed on the rock. The criteria may be, for example, similar to the criteria described with respect to element 506 or elsewhere herein.

The technique may further include outputting, at 606 based on the parameter of the rock and the criteria of the cooling process, a visual indication of an aspect of the cooling process. The visual indication may be, for example, one or more of the models depicted in FIGS. 2A, 2B, 3, and 4. More specifically, the aspect may be one or more of a transient temperature contour around the borehole during a cooling process, a temperature gradient contour around the borehole during a cooling process, induced axial stress along the axis of a borehole during a cooling process, or some other aspect.

It will be understood that the examples of FIGS. 5 and 6 are intended as high-level examples of various techniques related to the present disclosure, and in other embodiments the techniques may have more or fewer elements than are depicted. In some embodiments, certain of the elements (e.g., elements 602 and 604) may be performed in an order different than depicted, or concurrently with one another. Other variations may be present in other embodiments.

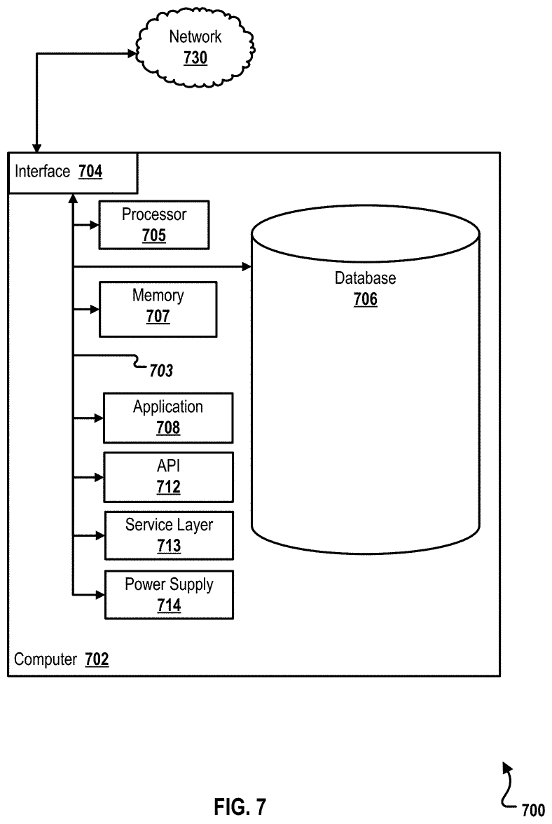

FIG. 7 is a block diagram of an example computer system 700 used to provide computational functionalities associated with described algorithms, methods, functions, processes, flows, and procedures described in the present disclosure, according to some implementations of the present disclosure. The illustrated computer 702 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 702 can include input devices such as keypads, keyboards, and touch screens that can accept user information. Also, the computer 702 can include output devices that can convey information associated with the operation of the computer 702. The information can include digital data, visual data, audio information, or a combination of information. The information can be presented in a graphical UI (or GUI).

The computer 702 can serve in a role as a client, a network component, a server, a database, a persistency, or components of a computer system for performing the subject matter described in the present disclosure. The illustrated computer 702 is communicably coupled with a network 730. In some implementations, one or more components of the computer 702 can be configured to operate within different environments, including cloud-computing-based environments, local environments, global environments, and combinations of environments.

At a top level, the computer 702 is an electronic computing device operable to receive, transmit, process, store, and manage data and information associated with the described subject matter. According to some implementations, the computer 702 can also include, or be communicably coupled with, an application server, an email server, a web server, a caching server, a streaming data server, or a combination of servers.

The computer 702 can receive requests over network 730 from a client application (for example, executing on another computer 702). The computer 702 can respond to the received requests by processing the received requests using software applications. Requests can also be sent to the computer 702 from internal users (for example, from a command console), external (or third) parties, automated applications, entities, individuals, systems, and computers.

Each of the components of the computer 702 can communicate using a system bus 703. In some implementations, any or all of the components of the computer 702, including hardware or software components, can interface with each other or the interface 704 (or a combination of both) over the system bus 703. Interfaces can use an application programming interface (API) 712, a service layer 713, or a combination of the API 712 and service layer 713. The API 712 can include specifications for routines, data structures, and object classes. The API 712 can be either computer-language independent or dependent. The API 712 can refer to a complete interface, a single function, or a set of APIs.

The service layer 713 can provide software services to the computer 702 and other components (whether illustrated or not) that are communicably coupled to the computer 702. The functionality of the computer 702 can be accessible for all service consumers using this service layer. Software services, such as those provided by the service layer 713, can provide reusable, defined functionalities through a defined interface. For example, the interface can be software written in JAVA, C++, or a language providing data in extensible markup language (XML) format. While illustrated as an integrated component of the computer 702, in alternative implementations, the API 712 or the service layer 713 can be stand-alone components in relation to other components of the computer 702 and other components communicably coupled to the computer 702. Moreover, any or all parts of the API 712 or the service layer 713 can be implemented as child or sub-modules of another software module, enterprise application, or hardware module without departing from the scope of the present disclosure.

The computer 702 includes an interface 704. Although illustrated as a single interface 704 in FIG. 7, two or more interfaces 704 can be used according to particular needs, desires, or particular implementations of the computer 702 and the described functionality. The interface 704 can be used by the computer 702 for communicating with other systems that are connected to the network 730 (whether illustrated or not) in a distributed environment. Generally, the interface 704 can include, or be implemented using, logic encoded in software or hardware (or a combination of software and hardware) operable to communicate with the network 730. More specifically, the interface 704 can include software supporting one or more communication protocols associated with communications. As such, the network 730 or the interface's hardware can be operable to communicate physical signals within and outside of the illustrated computer 702.

The computer 702 includes a processor 705. Although illustrated as a single processor 705 in FIG. 7, two or more processors 705 can be used according to particular needs, desires, or particular implementations of the computer 702 and the described functionality. Generally, the processor 705 can execute instructions and can manipulate data to perform the operations of the computer 702, including operations using algorithms, methods, functions, processes, flows, and procedures as described in the present disclosure.

The computer 702 also includes a database 706 that can hold data for the computer 702 and other components connected to the network 730 (whether illustrated or not). For example, database 706 can be an in-memory, conventional, or a database storing data consistent with the present disclosure. In some implementations, database 706 can be a combination of two or more different database types (for example, hybrid in-memory and conventional databases) according to particular needs, desires, or particular implementations of the computer 702 and the described functionality. Although illustrated as a single database 706 in FIG. 7, two or more databases (of the same, different, or combination of types) can be used according to particular needs, desires, or particular implementations of the computer 702 and the described functionality. While database 706 is illustrated as an internal component of the computer 702, in alternative implementations, database 706 can be external to the computer 702.

The computer 702 also includes a memory 707 that can hold data for the computer 702 or a combination of components connected to the network 730 (whether illustrated or not). Memory 707 can store any data consistent with the present disclosure. In some implementations, memory 707 can be a combination of two or more different types of memory (for example, a combination of semiconductor and magnetic storage) according to particular needs, desires, or particular implementations of the computer 702 and the described functionality. Although illustrated as a single memory 707 in FIG. 7, two or more memories 707 (of the same, different, or combination of types) can be used according to particular needs, desires, or particular implementations of the computer 702 and the described functionality. While memory 707 is illustrated as an internal component of the computer 702, in alternative implementations, memory 707 can be external to the computer 702.

The application 708 can be an algorithmic software engine providing functionality according to particular needs, desires, or particular implementations of the computer 702 and the described functionality. For example, application 708 can serve as one or more components, modules, or applications. Further, although illustrated as a single application 708, the application 708 can be implemented as multiple applications 708 on the computer 702. In addition, although illustrated as internal to the computer 702, in alternative implementations, the application 708 can be external to the computer 702.

The computer 702 can also include a power supply 714. The power supply 714 can include a rechargeable or non-rechargeable battery that can be configured to be either user- or non-user-replaceable. In some implementations, the power supply 714 can include power-conversion and management circuits, including recharging, standby, and power management functionalities. In some implementations, the power supply 714 can include a power plug to allow the computer 702 to be plugged into a wall socket or a power source to, for example, power the computer 702 or recharge a rechargeable battery.

There can be any number of computers 702 associated with, or external to, a computer system containing computer 702, with each computer 702 communicating over network 730. Further, the terms “client,” “user,” and other appropriate terminology can be used interchangeably, as appropriate, without departing from the scope of the present disclosure. Moreover, the present disclosure contemplates that many users can use one computer 702 and one user can use multiple computers 702.

Described implementations of the subject matter can include one or more features, alone or in combination.

For example, in a first implementation, a computer-implemented method includes identifying, by an electronic device, a parameter of rock adjacent to a stage of a multi-stage borehole; identifying, by the electronic device, a criteria of a cooling process that is to be performed on the rock; and outputting, by the electronic device based on the parameter of the rock and the criteria of the cooling process, a visual indication of an aspect of the cooling process.

The foregoing and other described implementations can each, optionally, include one or more of the following features:

A first feature, combinable with any of the following features, where the parameter of the rock is related to thermal conductivity of the rock, thermal expansion of the rock, specific heat of the rock, a Young's modulus related to the rock, a Poisson ratio related to the rock, density of the rock, axial tensile stress of the rock, or tensile strength of the rock.

A second feature, combinable with any of the previous or following features, where the criteria of the cooling process is related to a type of cooling agent that is to be used during the cooling process.

A third feature, combinable with any of the previous or following features, where the criteria is related to a time period over which the cooling process is to be performed.

A fourth feature, combinable with any of the previous or following features, where the criteria is related to a pre-identified induced axial tensile stress.

A fifth feature, combinable with any of the previous or following features, where the pre-identified induced in axial tensile stress is based on a maximum surface treating pressure that is to be sustained by a wellhead of the borehole during a hydraulic fracturing of the rock.

A sixth feature, combinable with any of the previous or following features, where the aspect of the cooling process is related to a transient temperature contour that is to be induced by a cooling agent during the cooling process.

A seventh feature, combinable with any of the previous or following features, where the aspect of the cooling process is related to a temperature gradient contour of the rock during the cooling process.

An eighth feature, combinable with any of the previous or following features, where the aspect of the cooling process is related to a pre-identified change in axial stress that is to be induced along an axis of the borehole as a result of the cooling process.

In a second implementation, a non-transitory, computer-readable medium stores one or more instructions executable by a computer system to perform operations including identifying a stage of a multi-stage borehole; identifying a parameter of rock that is adjacent the multi-stage borehole; identifying, based on the parameter, a criteria of a cooling process that is to be performed on the rock; and outputting an indication of the criteria for use in performing the cooling process.

The foregoing and other described implementations can each, optionally, include one or more of the following features:

A first feature, combinable with any of the previous or following features, where the parameter of the rock is related to thermal conductivity of the rock, thermal expansion of the rock, specific heat of the rock, a Young's modulus related to the rock, a Poisson ratio related to the rock, density of the rock, axial tensile stress of the rock, or tensile strength of the rock.

A second feature, combinable with any of the previous or following features, where the identification of the parameter is based on prior analysis of one or more samples of the rock.

A third feature, combinable with any of the previous or following features, where the criteria is related to a type of cooling agent that is to be used during the cooling process.

A fourth feature, combinable with any of the previous or following features, where the criteria is related to a time period over which the cooling process is to be performed.

A fifth feature, combinable with any of the previous or following features, where the time period is greater than 10 minutes.

A sixth feature, combinable with any of the previous or following features, where the time period is based on thermal mechanical properties of the rock.

A seventh feature, combinable with any of the previous or following features, where the time period is based on an injection schedule of a cooling agent that is to be used for the cooling process.

An eighth feature, combinable with any of the previous or following features, where the criteria is related to a pre-identified induced axial stress.

A ninth feature, combinable with any of the previous or following features, where the pre-identified induced axial tensile stress is based on tensile strength of the rock and in-situ stress of the rock.

In a third implementation, a computer-implemented system includes one or more processors and a non-transitory computer-readable storage medium coupled to the one or more processors and storing programming instructions for execution by the one or more processors. The programming instructions instruct the one or more processors to perform operations including identifying a stage of a multi-stage borehole, where a hydraulic fracturing process is to be performed on rock adjacent the stage; identifying a parameter of the rock; identifying, based on the parameter, a criteria of a cooling process that is to be performed on the rock; and outputting an indication of the parameter for use in performing the cooling process.

The foregoing and other described implementations can each, optionally, include one or more of the following features:

A first feature, combinable with any of the previous or following features, where the instructions are further to cause the electronic device to output a visual indication of an aspect of the cooling process.

A second feature, combinable with any of the previous or following features, where the aspect of the cooling process is related to one of: a transient temperature contour that is to be induced by a cooling agent during the cooling process, a temperature gradient contour of the rock during the cooling process, and a change in axial stress that is to be induced along an axis of the borehole as a result of the cooling process.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. For example, the signal can be a machine-generated electrical, optical, or electromagnetic signal that is generated to encode information for transmission to a suitable receiver apparatus for execution by a data processing apparatus. The computer-storage medium can be a machine-readable storage device, a machine-readable storage substrate, a random or serial access memory device, or a combination of computer-storage mediums.

The terms “data processing apparatus,” “computer,” and “electronic computer device” (or equivalent as understood by one of ordinary skill in the art) refer to data processing hardware. For example, a data processing apparatus can encompass all kinds of apparatuses, devices, and machines for processing data, including by way of example, a programmable processor, a computer, or multiple processors or computers. The apparatus can also include special purpose logic circuitry including, for example, a central processing unit (CPU), a field-programmable gate array (FPGA), or an application-specific integrated circuit (ASIC). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with or without conventional operating systems, such as LINUX, UNIX, WINDOWS, MAC OS, ANDROID, or IOS.

A computer program, which can also be referred to or described as a program, software, a software application, a module, a software module, a script, or code, can be written in any form of programming language. Programming languages can include, for example, compiled languages, interpreted languages, declarative languages, or procedural languages. Programs can be deployed in any form, including as stand-alone programs, modules, components, subroutines, or units for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files storing one or more modules, sub-programs, or portions of code. A computer program can be deployed for execution on one computer or on multiple computers that are located, for example, at one site or distributed across multiple sites that are interconnected by a communication network. While portions of the programs illustrated in the various figures may be shown as individual modules that implement the various features and functionality through various objects, methods, or processes, the programs can instead include a number of sub-modules, third-party services, components, and libraries. Conversely, the features and functionality of various components can be combined into single components as appropriate. Thresholds used to make computational determinations can be statically, dynamically, or both statically and dynamically determined.

The methods, processes, or logic flows described in this specification can be performed by one or more programmable computers executing one or more computer programs to perform functions by operating on input data and generating output. The methods, processes, or logic flows can also be performed by, and apparatus can also be implemented as, special purpose logic circuitry, for example, a CPU, an FPGA, or an ASIC.

Computers suitable for the execution of a computer program can be based on one or more of general and special purpose microprocessors and other kinds of CPUs. The elements of a computer are a CPU for performing or executing instructions and one or more memory devices for storing instructions and data. Generally, a CPU can receive instructions and data from (and write data to) a memory.

Graphics processing units (GPUs) can also be used in combination with CPUs. The GPUs can provide specialized processing that occurs in parallel to processing performed by CPUs. The specialized processing can include artificial intelligence (AI) applications and processing, for example. GPUs can be used in GPU clusters or in multi-GPU computing.

A computer can include, or be operatively coupled to, one or more mass storage devices for storing data. In some implementations, a computer can receive data from, and transfer data to, the mass storage devices including, for example, magnetic, magneto-optical disks, or optical disks. Moreover, a computer can be embedded in another device, for example, a mobile telephone, a personal digital assistant (PDA), a mobile audio or video player, a game console, a global positioning system (GPS) receiver, or a portable storage device such as a universal serial bus (USB) flash drive.

Computer-readable media (transitory or non-transitory, as appropriate) suitable for storing computer program instructions and data can include all forms of permanent/non-permanent and volatile/non-volatile memory, media, and memory devices. Computer-readable media can include, for example, semiconductor memory devices such as random access memory (RAM), read-only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Computer-readable media can also include, for example, magnetic devices such as tape, cartridges, cassettes, and internal/removable disks. Computer-readable media can also include magneto-optical disks and optical memory devices and technologies including, for example, digital video disc (DVD), CD-ROM, DVD+/−R, DVD-RAM, DVD-ROM, HD-DVD, and BLU-RAY. The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated into, special purpose logic circuitry.

Implementations of the subject matter described in the present disclosure can be implemented on a computer having a display device for providing interaction with a user, including displaying information to (and receiving input from) the user. Types of display devices can include, for example, a cathode ray tube (CRT), a liquid crystal display (LCD), a light-emitting diode (LED), and a plasma monitor. Display devices can include a keyboard and pointing devices including, for example, a mouse, a trackball, or a trackpad. User input can also be provided to the computer through the use of a touch-screen, such as a tablet computer surface with pressure sensitivity or a multi-touch screen using capacitive or electric sensing. Other kinds of devices can be used to provide for interaction with a user, including to receive user feedback including, for example, sensory feedback including visual feedback, auditory feedback, or tactile feedback. Input from the user can be received in the form of acoustic, speech, or tactile input. In addition, a computer can interact with a user by sending documents to, and receiving documents from, a device that the user uses. For example, the computer can send web pages to a web browser on a user's client device in response to requests received from the web browser.

The term “graphical user interface,” or “GUI,” can be used in the singular or the plural to describe one or more graphical user interfaces and each of the displays of a particular graphical user interface. Therefore, a GUI can represent any graphical user interface, including, but not limited to, a web browser, a touch-screen, or a command line interface (CLI) that processes information and efficiently presents the information results to the user. In general, a GUI can include a plurality of UI elements, some or all associated with a web browser, such as interactive fields, pull-down lists, and buttons. These and other UI elements can be related to or represent the functions of the web browser.

Implementations of the subject matter described in this specification can be implemented in a computing system that includes a back-end component, for example, as a data server, or that includes a middleware component, for example, an application server. Moreover, the computing system can include a front-end component, for example, a client computer having one or both of a graphical user interface or a web browser through which a user can interact with the computer. The components of the system can be interconnected by any form or medium of wireline or wireless digital data communication (or a combination of data communication) in a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) (for example, using 802.11 a/b/g/n or 802.20 or a combination of protocols), all or a portion of the Internet, or any other communication system or systems at one or more locations (or a combination of communication networks). The network can communicate with, for example, Internet Protocol (IP) packets, frame relay frames, asynchronous transfer mode (ATM) cells, voice, video, data, or a combination of communication types between network addresses.

The computing system can include clients and servers. A client and server can generally be remote from each other and can typically interact through a communication network. The relationship of client and server can arise by virtue of computer programs running on the respective computers and having a client-server relationship.

Cluster file systems can be any file system type accessible from multiple servers for read and update. Locking or consistency tracking may not be necessary since the locking of exchange file system can be done at application layer. Furthermore, Unicode data files can be different from non-Unicode data files.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations. It should be understood that the described program components and systems can generally be integrated together in a single software product or packaged into multiple software products.

Accordingly, the previously described example implementations do not define or constrain the present disclosure. Other changes, substitutions, and alterations are also possible without departing from the spirit and scope of the present disclosure.

Furthermore, any claimed implementation is considered to be applicable to at least a computer-implemented method; a non-transitory, computer-readable medium storing computer-readable instructions to perform the computer-implemented method; and a computer system including a computer memory interoperably coupled with a hardware processor configured to perform the computer-implemented method or the instructions stored on the non-transitory, computer-readable medium.

What is claimed is:

1. One or more non-transitory computer-readable media comprising instructions that, upon execution of the instructions by a processor of an electronic device, are to cause the electronic device to:
identify a stage of a multi-stage borehole;
identify a parameter of rock that is adjacent the multi-stage borehole;
identify, based on the parameter, a criteria of a cooling process that is to be performed on the rock; and
output an indication of the criteria for use in performing the cooling process.

2. The one or more non-transitory computer-readable media of claim 1, wherein the parameter of the rock is related to thermal conductivity of the rock, thermal expansion of the rock, specific heat of the rock, a Young's modulus related to the rock, a Poisson ratio related to the rock, density of the rock, axial tensile stress of the rock, or tensile strength of the rock.

3. The one or more non-transitory computer-readable media of claim 1, wherein the identification of the parameter is based on prior analysis of one or more samples of the rock.

4. The one or more non-transitory computer-readable media of claim 1, wherein the criteria is related to a type of cooling agent that is to be used during the cooling process.

5. The one or more non-transitory computer-readable media of claim 1, wherein the criteria is related to a time period over which the cooling process is to be performed.

6. The one or more non-transitory computer-readable media of claim 5, wherein the time period is greater than 10 minutes.

7. The one or more non-transitory computer-readable media of claim 1, wherein the criteria is related to a pre-identified induced axial tensile stress.

8. The one or more non-transitory computer-readable media of claim 7, wherein the pre-identified induced axial tensile stress is based on tensile strength of the rock and in-situ stress of the rock.

9. A method comprising:
identifying, by an electronic device, a parameter of rock adjacent to a stage of a multi-stage borehole;
identifying, by the electronic device, a criteria of a cooling process that is to be performed on the rock; and
outputting, by the electronic device based on the parameter of the rock and the criteria of the cooling process, a visual indication of an aspect of the cooling process.

10. The method of claim 9, wherein the parameter of the rock is related to thermal conductivity of the rock, thermal expansion of the rock, specific heat of the rock, a Young's modulus related to the rock, a Poisson ratio related to the rock, density of the rock, axial tensile stress of the rock, or tensile strength of the rock.

11. The method of claim 9, wherein the criteria of the cooling process is related to a type of cooling agent that is to be used during the cooling process.

12. The method of claim 9, wherein the criteria is related to a time period over which the cooling process is to be performed.

13. The method of claim 9, wherein the criteria is related to a pre-identified induced axial tensile stress.

14. The method of claim 13, wherein the pre-identified induced in axial tensile stress is based on a maximum surface treating pressure that is to be sustained by a wellhead of the borehole during a hydraulic fracturing of the rock.

15. The method of claim 9, wherein the aspect of the cooling process is related to a transient temperature contour that is to be induced by a cooling agent during the cooling process.

16. The method of claim 9, wherein the aspect of the cooling process is related to a temperature gradient contour of the rock during the cooling process.

17. The method of claim 9, wherein the aspect of the cooling process is related to a pre-identified change in axial stress that is to be induced along an axis of the borehole as a result of the cooling process.

18. An electronic device comprising:
one or more processors; and
one or more non-transitory computer-readable media comprising instructions that, upon execution of the instructions by the one or more processors, are to cause the electronic device to:
identify a stage of a multi-stage borehole, wherein a hydraulic fracturing process is to be performed on rock adjacent the stage;
identify a parameter of the rock;
identify, based on the parameter, a criteria of a cooling process that is to be performed on the rock; and
output an indication of the parameter for use in performing the cooling process.

19. The electronic device of claim 18, wherein the instructions are further to cause the electronic device to output a visual indication of an aspect of the cooling process.

20. The electronic device of claim 19, wherein the aspect of the cooling process is related to one of: a transient temperature contour that is to be induced by a cooling agent during the cooling process, a temperature gradient contour of the rock during the cooling process, and a change in axial stress that is to be induced along an axis of the borehole as a result of the cooling process.
